(19) United States
(12) Patent Application Publication
(10) Pub. No.: US 2015/0314118 A1
(43) Pub. Date: Nov. 5, 2015

(54) SYSTEM, APPARATUS AND METHOD FOR ESTABLISHING INTRAOSSEOUS VASCULAR ACCESS

(71) Applicant: Benvenue Medical, Inc., Santa Clara, CA (US)

(72) Inventors: Alexios Kelekis, Athens (GR); Laurent Schaller, Los Altos, CA (US)

(21) Appl. No.: 14/700,366

(22) Filed: Apr. 30, 2015

Related U.S. Application Data
(60) Provisional application No. 61/987,107, filed on May 1, 2014.

(51) Int. Cl. A61M 39/02; A61M 39/04; A61B 17/34

(52) U.S. Cl. CPC A61M 39/02; A61M 39/0208; A61B 17/3472; A61M 39/04; A61M 2039/0205; A61M 2039/0232; A61M 2039/0202

(57) ABSTRACT

System, Apparatus and Method provide intraosseous access to the systemic venous system of a subject. The system includes a bone access device with a drug discharge aperture for implantation, a port including a needle-penetrable septum for subcutaneous placement and a fluid flow path connecting the port and access device.

### RELATED APPLICATION INFORMATION

The present application claims priority to and the benefit of U.S. Provisional Patent Application Ser. No. 61/987107, filed May 1, 2014, which is hereby incorporated by reference.

### BACKGROUND

The present application generally relates to intraosseous access and, more particularly, to systems, apparatus and methods for establishing and using intraosseous vascular access to the systemic venous system of a subject.

“Intraosseous” generally refers to inside or within a bone or bony structure. There are many clinical conditions where it is helpful to provide intraosseous access. In some cases it may be necessary to treat diseases with bone marrow or stem cell transplants to restore functioning blood cells. Such conditions may include, but are not limited to, acute leukemia, brain tumors, breast cancer, Hodgkin's disease, multiple myeloma, neuroblastoma, non-Hodgkin's lymphomas, ovarian cancer, sarcoma and testicular cancer. In other cases it may be necessary to access bone marrow to obtain a sample or specimen of the marrow for diagnostic testing. These conditions may include, but are not limited to, cancers of any type and hematologic disease of any origin.

Intraosseous access also may necessary or desirable for vascular or venous access. The use of an intraosseous route or avenue for venous access was first introduced by Drinker in 1922 as a method for accessing non-collapsible venous plexuses (vascular networks) through the bone marrow cavity to systemic circulation. The development of intravenous catheters supplanted this technique until the 1980s, when intraosseous access was reintroduced—particularly for rapid fluid infusion during resuscitation.

Intrasosseous access has been suggested for children aged 6 years or younger. Recent studies have shown that it also is safe in older children and adults. Successful infusions in newborns have further suggested that access via the intraosseous route is faster than access via umbilical veins. According to the Emergency Cardiovascular Care Guidelines in 2000, intraosseous access is recommended in all children after two failed attempts of intravenous access or during circulatory collapse.

In 2005, the American Heart Association recommended intraosseous access if venous access cannot be quickly and reliably established, but heart-related emergencies are not the only situation where quick venous access is needed. Every year, millions of patients are treated for life-threatening emergencies in the United States. Such emergencies include shock, trauma, drug overdoses, diabetic ketoacidosis, arrhythmias, burns, and status epilepticus just to name a few. An essential element for treating all such emergencies may be the rapid establishment of an intravenous (IV) line in order to administer drugs and fluids directly into the circulatory system. Whether in the ambulance by paramedics, or in the emergency room by emergency specialists, the goal is the same—to start an IV in order to administer life-saving drugs and/or fluids.

While it is relatively easy to start an IV on some patients, doctors, nurses and paramedics often experience great difficulty establishing IV access in approximately 20 percent of patients. These patients are probed repeatedly with sharp needles in an attempt to solve this problem and may require an invasive procedure to finally establish an intravenous route. A further complicating factor in achieving IV access occurs “in the field” e.g. at the scene of an accident, combat injury or during ambulance transport, where it is difficult to see or find the target vein and excessive motion makes a successful venipuncture very difficult.

In other cases, such as patients with chronic disease or the elderly, the availability of easily-accessible veins may be depleted. Other patients may have no available IV sites due to anatomical scarcity of peripheral veins, obesity, extreme dehydration or previous IV drug use. For these patients, finding a suitable site for administering lifesaving drugs can become a frustrating task. While morbidity and mortality statistics are not generally available, it is understood that patients with life-threatening emergencies may have died of ensuing complications because access to the vascular system with life-saving IV therapy was delayed or simply not possible. For such patients, an alternative approach is required.

It has been said that intraosseous access may be easily established by users with little training and is more rapidly achieved than intravenous access. On the other hand, gaining access to bone and associated bone marrow for a small biopsy specimen or aspiration of a larger quantity of bone marrow has been said to sometimes be difficult, traumatic and occasionally dangerous, depending on each selected target area for harvesting bone and/or associated bone marrow, operator expertise and patient anatomy.

Manual insertion with force has been a primary techniques for introsseous access. For example, currently available devices and techniques for gaining access to a bone and the associated cancellous bone or a bone marrow cavity or space may include an intraosseous (IO) needle with a removable trocar disposed therein. Various shapes and sizes of handles may be used to apply manual pressure and to manually rotate the IO needle and removable trocar as a set. Such manual IO devices often require substantial force to break through the outer cortex of a bone. Exertion of such force may cause pain to a patient and may sometimes damage the bone and/or IO device. Such force may cause damage when harvesting bone marrow from children with softer bone structures or any patient with bones deteriorated by disease (cancer). Understandably, automated intraosseous insertion devices such as the EZ-IO (Vidacare Corp, San Antonio, Tex.), have recently gained popularity, and studies appear to have suggested these automated devices are safe and successful on first attempts in both children and adults.

However, there continues to be a need for alternative devices and methods for providing intraosseous access to the venous system, particularly for longer term or chronic treatments.

### BRIEF DESCRIPTION OF THE FIGURES

Turning now to a more detailed description of the present subject matter, system, apparatus, method and components.

FIG. 1 is a perspective representation of one example of an intraosseus access system of the present subject matter for systemic drug treatment delivery.

FIG. 2 is a perspective view of one configuration of bone access device or member in the form of a helical structure with fluid flow ports and passageway that may be used in the present subject matter, as illustrated in FIG. 1.

FIG. 3 is a perspective view of another configuration of bone access device or member in the form of a generally straight or slightly curved implant with a fluid flow path and slots that may be used in the present subject matter.

FIG. 4 is a perspective view of yet a further configuration of bone access device or member in the form of a straight screw type device with side holes, helical external raised rib or thread, and fluid passageway.

FIG. 5 is perspective view of a still further configuration of bone access device or member in the form of a straight structure with side holes, fluid pathway and a series of truncated conical surface elements that provide retention features or barbs.

FIG. 6a is a side view of a direct anchoring bone access device or member in the form of a threaded structure or bone anchor.

FIG. 6b is an end view of the anchor of FIG. 6a.

FIG. 7 is a side view of a bone access device or member similar to FIG. 6a but with a different side hole arrangement for injection.

FIG. 8 is a representation of fluid flow tubing being secured to a bone access device or member, such as shown in FIG. 6a or 7, which is shown in cross-section.

FIG. 9 is a perspective view of a flexible implantable catheter-like fluid flow tube that provides a fluid flow path between a bone access device or member (not seen) and a subcutaneous access port or reservoir for fluid introduction.

FIG. 10 is a representation of a system, including a bone access device or member, a subcutaneous port or reservoir and fluid flow tube or line connecting the device and port, providing access to a vertebral body for treatment of metastases in the spine, for example.

FIG. 11 is a representation of one form of a system employing a bone access device or member having a threaded external configuration for accessing a bone such as a femur or tibia, for instance, the bone access device or member being a screw-in type device with side holes for injection, and fluid flow line extending from the device to a port, such as a subcutaneous port, not shown.

FIG. 12 is a representation of an example of a push-in type bone access device or member with retention barbs and fluid flow path and side holes for injection and a fluid flow line or tube extending between the device and a port, such as a subcutaneous port, not shown.

FIG. 13 is perspective view of the push-in type bone access device or member with a fluid flow path and side ports such as seen in FIG. 12.

FIGS. 14a-14b are, respectively, horizontal and vertical cross-sectional views of a vertebral body, illustrating one prior technique and associated apparatus for introducing a distraction device into a vertebral body, which may be employed to introduce a bone access device into the vertebral body or elsewhere in the skeletal system for intraosseous access.

FIGS. 15a-15d are perspective views of a vertebral body, illustrating another prior technique and associated apparatus for introducing a distraction device into a vertebral body with the assistance of a guide wire, which may be employed to introduce a bone access device into a vertebral body or other bone for intraosseous access.

### DETAILED DESCRIPTION

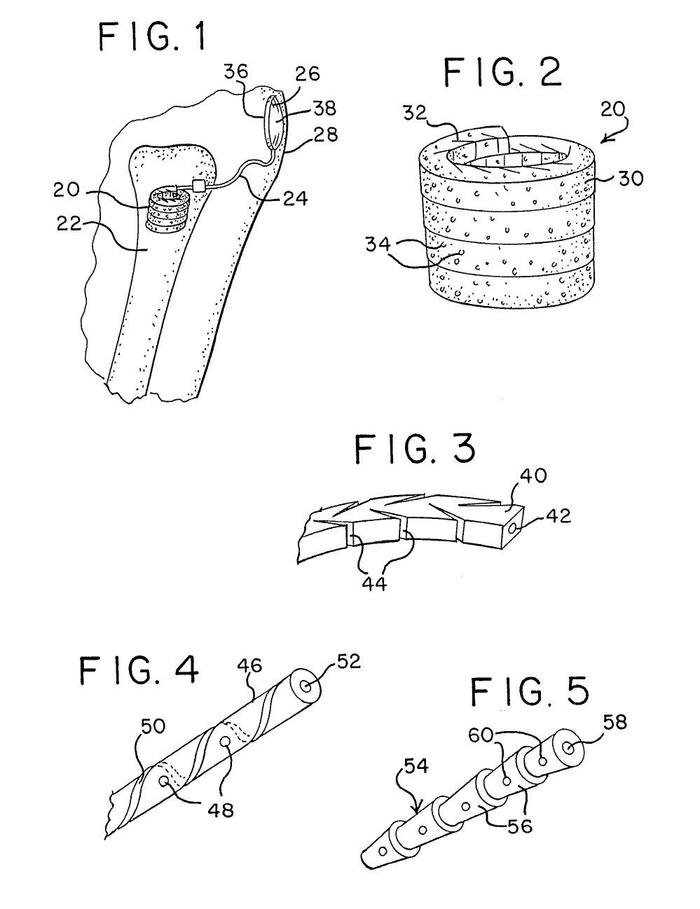

For intraosseous access, one embodiment of the present subject matter employs apparatus and insertion methods described in one or more of the following patent applications, which are incorporated by reference herein in their entirety: published PCT applications WO/2007/022194; WO/2008/103781 and WO/2012/064817. A commercial version of such apparatus is referred to as the Kiva VCF Treatment System from Benvenue Medical, Inc. of Santa Clara, Calif., USA. More particularly, the Kiva device, which has a helical implanted shape similar to that seen in FIGS. 1 and 2, is employed in the present subject matter as bone access device or member for systemic drug treatment delivery.

As used herein, “drug” has a comprehensive meaning and includes medical injectables of any type, including without limitation antibiotics, chemotherapy drugs, biologics, saline, blood and blood products, insulin and any other medical fluids for therapeutic or diagnostic purposes typically associated with intravenous therapy.

In accordance with one aspect of the present subject matter, referring to FIG. 1, a bone access device or member 20 is implanted into a desired location within a bone 22, such as marrow-containing space or cavity or a region of cancellous bone. Fluid flow tube or line 24 extends between and connects the implant 20 and a subcutaneous port or reservoir 26 for ease of access through skin surface 28 for one time or recurring drug injection to facilitate therapeutic or diagnostic treatments within the body of the patient.

More specifically, the particular configuraton of the bone access device or member 20 illustrated in FIG. 1 and FIG. 2 is similar to the Benvenue Kiva device identified above. The device comprises an elongated member or element 30 made of PEEK or other suitable material that is introduced into the bone in a generally linear configuration, typically through an introduction cannula and optionally also over a guidewire, and forms in situ (within bone 22) into a helical configuration as seen in FIGS. 1 and 2. The device 20 has an elongated central passageway, not shown in FIGS. 1 and 2, that extends axially along the length of the member between the proximal and distal ends and communicates through one or more side apertures or openings in the wall of the member for flowing drugs into the bone. In the commercial Kiva device the openings are in the form of slots that also facilitate curving or bending of the element into the helical configuration. As shown in FIG. 2, the openings are in the form of internal slits or slots 32 and small ports 34 located along the length of the implant.

Turning back to FIG. 1, the system shown there also includes the subcutaneous port or reservoir 26 and flexible fluid flow tube 24 that extends between the port or reservoir 26 and the passageway of the implant. Subcutaneous access ports or reservoirs have been used heretofore in a variety of prior applications, and the port or reservoir 26 may be of conventional construction. It may be a port-only device for receiving a transcutaneous injection of drugs, which flow directly to the implant 20. Alternatively, the port 26 may have a reservoir, such as an expandable resilient elastomeric chamber or bladder that can be periodically filled and/or refilled transcutaneously, and gradually expel drug under pressure from the elastomeric chamber to the implant 20 and through the implant into the bone tissue for uptake into the systemic venous system of the patient. For purposes of this description, unless specified otherwise, the term “port” will be used generically to include both port-only structures and ports that include reservoirs.

As seen in FIG. 1, the access port 26 is of conventional construction and has a needle-impenetrable housing 36 that may enclose a fluid reservoir that is accessible from the exterior of the access port through a needle-penetrable elastomeric septum 38. The port 26 also includes an outlet that is connected to fluid flow tube 24 for the flow of drug from the port to the bone access device or member 20. It should be noted that the port can provide two-way intraosseous access, allowing the use of a needle-syringe for transcutaensous withdrawal or aspiration of fluid, such as blood, bone marrow or other tissue, through the implant 20, fluid conduit 24 and port 26. Blood obtained through intraosseous access may be used to obtain most laboratory values, including pH level, PCO₂ level, and ABO and Rh typing, although it is known that the results of these standard laboratory tests may differ slightly from results obtained with venous blood.

For an implanted vascular access port 26 to be successful in long term implantation or treatment applications, the septum 38 of the vascular access port is preferably but not exclusively possessed of specific properties. The subcutaneous placement of a vascular access port makes it difficult to predict with precision the location in cross section of the septum of that vascular access port that will be penetrated by a hypodermic needle on any given occasion. The septum installed in the vascular access port should thus exhibit substantially uniform needle sealing, needle retention, and needle penetration characteristics across the entire area of the septum exposed to needle penetration. In this manner, the quality of the interaction between a septum and the shaft of a penetrating hypodermic needle will be substantially independent of the location at which the tip of the hypodermic needle actually enters the septum.

A relatively large needle target area for the port septum is desirable for various reasons. Missing the needle target area of the septum 38 of vascular access port 26 may be a painful event for the patient. It is an event that also presents major risks. If the miss is not detected by medical personnel, the fluids in the associated hypodermic syringe could be injected subcutaneously into the subcutaneous region or pocket in which the vascular access port is implanted, producing potentially adverse consequences or reducing the effectiveness of the drug injected.

A large needle target area in the septum 38 of the vascular access port 26 also decreases the likelihood that the desirable repeated selective penetration of the septum by the tip of a hypodermic needle will inadvertently become concentrated over time in any small region of the septum. The dispersal of puncture sites over a large needle target area slows the destructive effects of needle penetration, such as septum coring, and thus contributes to septum and port longevity.

Accordingly, with the combination of a bone access device 20, subcutaneous access port 26 and connecting fluid flow line 24, the present subject matter provides a way to anchor a bone access device, establish a pathway for access to the systemic venous system via the bone tissue, and provide a system suitable for repeated injection (and/or aspiration) for on-going treatment over an extended period of time. Such combinations or systems can be implanted in many different locations in the skeletal system, such as the vertebral body (VB), the iliac crest, the pelvic, the femur, the shoulder blade and long bones to just name a few.

The disclosed system, apparatus and method of the present application helps avoid potential issues with vascular access systems such as pinch-off (or occlusion) and injuries to the vascular system, as well as concerns that can arise with conventional intraosseous needles that extend through the skin into bone. Such intraosseous needles can create a higher risk of infection if left in the bone for longer than 72 hours. With the present system, essentially all suitable medications, medical fluids, drugs and blood products can be safely administered through the intraosseous path, and the onset of action and peak drug levels are understood to be at least comparable to those of intravenous administration.

As described in more detail below, there are shown several additional and non-exclusive configurations of the bone access devices or members (which may also be referred to as bone anchors or bone plugs or by similar terms) that may be implanted as part of the present system and method. Specifically, FIGS. 3-5 show alternative bone access implant devices or members. In. FIG. 3, the bone access device or member 40 is generally a straight or slightly curved construction, with an internal elongated flow path 42 that extends longitudinally through the device, and side slots or slits 44 that are illustrated as angled relative to the longitudinal axis. The slots or slits 44 intercept the flow path 42 to provide passageways that distribute drugs into intraosseous tissue as they are introduced into the access device from a subcutaneous port. The device 40 may include a connector (not shown) of suitable configuration, such as a luer or luer lock, for attachment to a flow tube or line extending from the port. Alternatively, the access device may be bonded or otherwise permanently attached to such tubing. These alternative connection arrangements also apply to the other bone access devices or members described below.

FIG. 4 shows a straight, screw-type bone access implant device 46 with side holes 48 and a raised helical rib or thread 50 on the exterior surface for engaging and anchoring or retaining the device in the bone. The side holes open into an elongated internal flow path 52 that extends the length of the device to distribute drug into surrounding bone tissue, such as bone marrow or cancellous bone, when implanted.

FIG. 5 shows a straight bone access device or member 54 with an exterior surface having retention structures in the form of a series truncated conical surface features 56, in cross-section similar to a saw-tooth shape or barbs, for engaging bony tissue and retaining or anchoring the device. The device 54 also has an internal fluid flow path or bore 58 extending along its length and connecting to side apertures or openings 60 to distribute drug to the bone tissue when implanted.

The bone access device or member could also be configured as illustrated in FIGS. 6-8. FIGS. 6a and 6b show a direct anchoring threaded bone access device or member 62 with a tapered or pointed insertion end 64, a slotted opposite end 66, an external raised helical rib or thread 68 for screw-type insertion and anchoring within bone tissue, an internal flow passageway or lumen 70 and side holes or apertures 72 for drug injection into the bone.

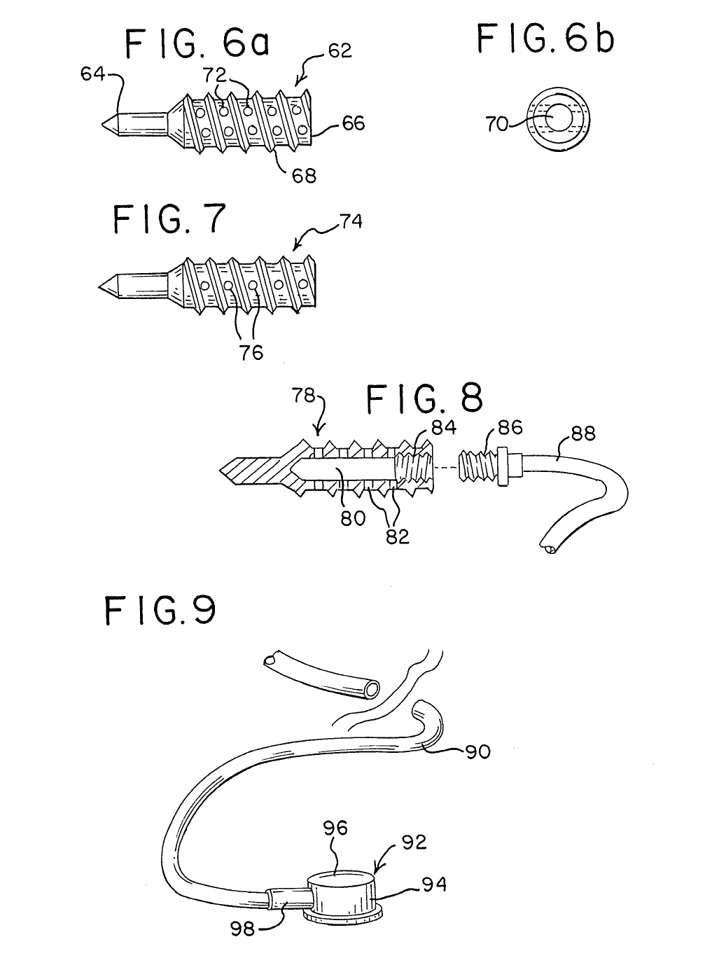

The bone access device or member 74 in FIG. 7 is similar to FIG. 6a, but with a different arrangement of apertures 76.

FIG. 8 is a cross-section view of a bone access device or member 78, that could be identical to the device of either FIGS. 6a-6b or 7. FIG. 8 better illustrates an internal flow path or lumen 80 that communicates through side apertures 82 and has a proximal threaded connecter end 84 for attachment to threaded end connector 86 of flexible, e.g. plastic or latex, fluid flow tubing 88 that extends from a port (not shown). The flow tubing connector 86 and connection end 84 of device 78 may be connected after insertion or implantation of the access device 78 into a suitable intra-bone location. The flow tubing is preferably inserted or implanted along a pathway that prevents occlusion, pinch-off or kink, and can be pre-attached or attachable to a subcutaneous port of conventional construction as illustrated in FIG. 9.

Turning to FIG. 9, shown there is exemplary flexible fluid flow tubing 90 attached to a subcutaneous port 92 of conventional construction. The port 92 includes a needle-impermeable base or housing 94, a needle penetrable septum 96 and a flow port 98 attached or attachable to tubing 90. The tubing and port can be attached to a bone access device or member before or after insertion or implantation, and the attachment can be permanent or removable.

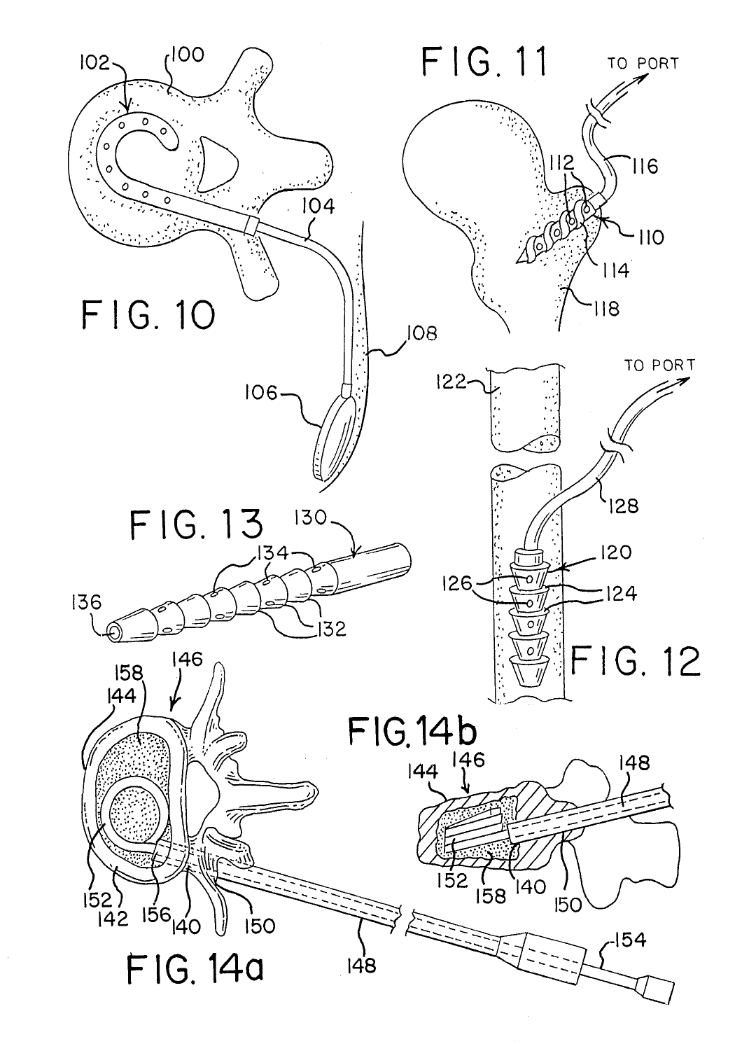

Still other examples of such systems for intraosseous venous access are described below. FIG. 10 illustrates one example of the present system for access to a vertebral body for treatment of metastases in the spine for instance. In FIG. 10 a curved implant bone access device 102 (pig-tail like design) is inserted into a vertebral body 100 via a transpedicular access similar to that used in balloon kyphoplasty or insertion of the Kiva device. As noted earlier, this entails providing access through the cortical wall of the vertebral body using known tools and steps. A cannula is inserted through the cortical wall and the implant 102 is inserted, typically in a straight configuration, through the cannula and into the inside of the vertebral body between the vertebral body endplates, where it curves in situ into the implanted shape. The implant 102 may or may not be delivered over a guide wire that has previously been advanced into the vertebral body, and may be inherently biased, as by a pre-shaping or heat setting, for example, to assume the desired shape upon exit from the introduction cannula into the vertebral body.

After the implant is 102 is inserted into the vertebral body 100, if not previously attached, one end of a flexible connecting line or tube 104 is attached to the proximal end of the implant. The tubing is in fluid flow communication with a lumen extending along the length of the implant. The other end of the tube is connected to a subcutaneous port 106 that is implanted just below the surface of skin 108 of the patient. The implant can be made of different implantable material such as stainless steel, titanium or polymer material such as implantable polyetheretherketone (PEEK) polymer. Other materials may also be suitable.

In order to access other skeleton locations, such as long bone, femur or tibia for instance, different designs of bone access devices or members might be more appropriate. FIGS. 11 and 12 are examples of such designs. FIG. 11 shows a screw-in like access device 110 implanted into bone and with an internal lumen (not shown) communicating with side holes 112 for injection, raised external helical rib or thread 114 and fluid flow tubing 116 to a port (not seen). FIG. 11 illustrates the device 110 within a bony structure, such as a trochanter, of a femur 118 or similar bone.

FIG. 12 shows a push-in type access device 120, similar to FIG. 5, within a long bone 122. The push-in device 120 has retention barbs 124, and internal lumen and connecting side holes 126 for injection. Fluid flow tubing 128 extends between one end of the device, where it flows into the lumen, and a port (not shown in FIG. 12).

FIG. 13 shows details of the push-in type bone access device 130, similar to FIGS. 5 and 12, which has an outer surface having retention surfaces such as retention barbs 132, side holes 134 and a center lumen 136 that extends the length of the implant and is in fluid connection with side holes 134.

### METHOD

The present subject matter can be used as described below, which is a non-exclusive exemplary description. A first step will be the insertion or anchoring of the bone access device into a bony site using a standard approach with a Jamshidi access needle and a cannulated working channel or cannula suitable for the device insertion, such as illustrated in the published PCT patent applications incorporated by reference herein.

First a site needs to be determined based on the patient condition and where best to set the bone access device into the bone. For instance, if it would be beneficial to treat spinal metastases in the spine, the bone access device of choice would be the Kiva like structure as shown in FIGS. 1 and 2 or the pig-tailed like implant shown in FIG. 10. These may be deployed, preferably over a guide wire, using a transpedicular access. On the other hand, if there is a need for a pelvic or femoral treatment, a straight bone access device such as described above could be used.

The method to access the site would include the use of an access needle or Jamshidi under fluoroscopic guidance. Once the site is targeted, a Kirschner wire will be used to exchange the Jamshidi needle for a dilator and working channel or cannula in order to provide a working access to the cancellous bone portion (or a bone marrow cavity) of the targeted area.

Once the site is accessed, the bone access device will be advanced over a guide wire or unassisted directly through the working cannula and a pusher or rotator can be used to advance the bone access device. Based on the type of bone access device used, the pusher or inserter will have the capability to securely attach the bone access device into the bone by pushing, tapping, twisting or screwing the device. The pusher or inserter may have the mechanical advantage of a ratcheting mechanism or a screw type action or similar function.

After the bone access device is fully in place, implanted within the bone, the inserter can be removed. If the fluid flow tubing is not pre-attached, a flexible fluid flow tube of polyurethane, latex or other material suitable for long term implantation may be attached at the end of the access device and communicate with an internal fluid flow path in the device for drug injection. In that event, the implant device will be configured to connect to the tube. This connection, as noted earlier, can be of any suitable configuration and can be a push type connector, screw type or other combination suitable for implant connection.

After the connection between the implant and fluid flow tube is competed, if necessary, the working cannula is removed and the tubing length is sized for attachment at its proximal end to an implant port device that will be placed just under the skin for easy access, as illustrated for example in FIG. 10.

Turning to FIGS. 14a and 14b, those figures, respectively, are horizontal and vertical cross-sectional views of a vertebral body, illustrating one prior technique and associated apparatus (described in one or more of the published PCT applications incorporated by reference above) for introducing a distraction device into a vertebral body, which may be employed to introduce a bone access device into the vertebral body or elsewhere in the skeletal system for intraosseous access.

In a typical procedure for treatment of a vertebral body, access to the vertebra can be gained by using the same procedures and techniques that are used for the other skeletal locations mentioned above, or by any other procedures and techniques generally known by those skilled in the art. Referring to FIGS. 14a and 14b, which illustrate one potential procedure, an access opening 140 is drilled into the cortical rim 142 of the vertebral body 144 of a vertebra 146. Cannula 148 is inserted through the access hole 140 into the vertebral body 146. Alternatively, the cannula 148 may be placed adjacent to the access hole 140 instead of inserted through the access hole. Typically, the access opening 140 will be drilled through the pedicle 150, which is sometimes referred to as a transpedicular approach. However, the access hole 140 could be made in any other portion of the cortical rim 142 as the physician may chose.

An implant 152, which will be referred to as the bone access device or anchor for purposes of this description, may be prepositioned within the cannula 148, which constrains the distraction device in the deformed or pre-deployed generally straight configuration. As the pushrod 154 is advanced, the access device 152 is advanced out of the distal end portion 156 of the cannula 148 and into the cancellous bone 158 of the vertebral body 144. Upon exiting the cannula 148, the access device 152 will begin to revert, by change of configuration, to its initial or deployed coil helical shape. Thus, as it is advanced from the cannula, the access device 152 winds up or curves into the relatively spongy cancellous bone 158 of the vertebral body 144 as shown in FIG. 14b.

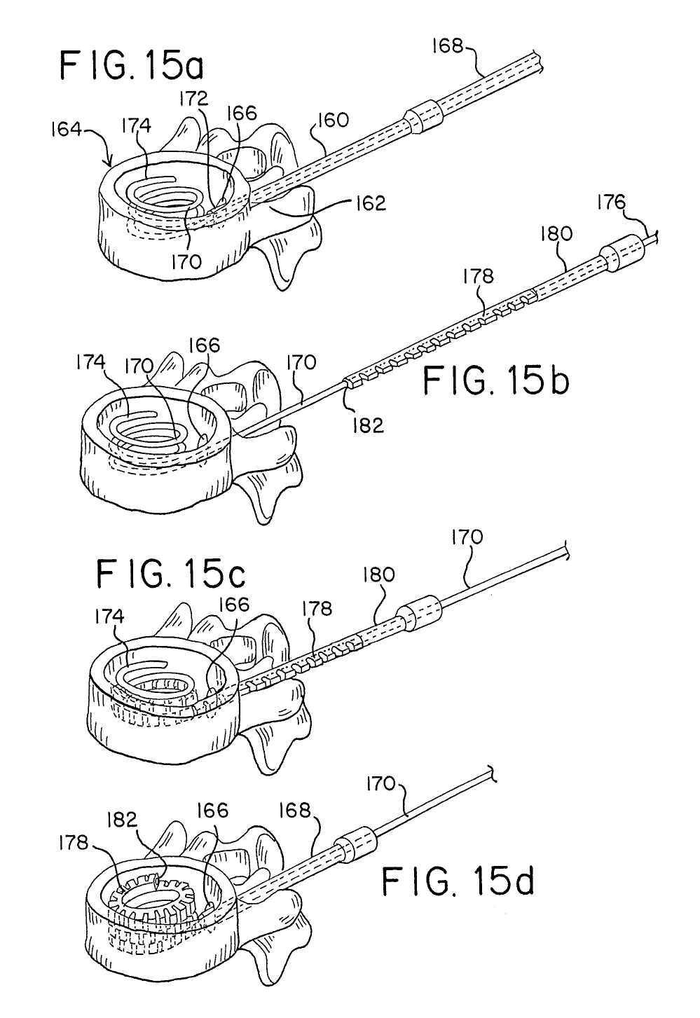

FIGS. 15a-15d are perspective views of a vertebral body, illustrating another prior technique and associated apparatus (described in one or more of the published PCT applications incorporated by reference above) for introducing a distraction device into a vertebral body with the assistance of a guide wire, which may be employed to introduce a bone access device into a vertebral body or other bone for intraosseous access. Referring to FIG. 15a, an introducer sheath 160 is introduced through the back of a patient while the patient is lying in a prone position. Fluoroscopic guidance using a biplane imaging system for better visualization of the spine may be used to help guide the delivery system to the desired location. The introducer sheath 160 has a sharp tip to help penetrate the bone structure typically through the pedicle 162 of the vertebral body 164 (in the transpedicular approach). Once the introducer sheath 160 has passed through or created a passage 166 in the pedicle 162 and is in the desired position, which can be confirmed by imaging, a delivery cannula 168 may be inserted into the introducer sheath 160 and a guide wire 170 is advanced forward through the cannula. Alternatively, the guide wire may be inserted through the cannula without an introducer sheath.

The guide wire 170 is preferably made of a shape memory material that has an initial or free state in the shape of a helical coil or spring. As the guide wire 170 is inserted into the cannula 168, the cannula constrains the guide wire into a generally elongated linear straight configuration, allowing an easy and minimally invasive deployment of the guide wire into the treatment site. Because of the shape memory properties, the guide wire 170 will return to its coil-shaped free state once the constraint is removed, i.e., as the guide wire exits the distal end portion 172 of the cannula 168 and enters the vertebral body 164. The guide wire 170 can be advanced through the cannula 164 manually or with the aid of an advancing mechanism.

As the guide wire 170 exits the distal end portion 172 of the cannula 168 and enters the vertebral body 164, the distal end portion 174 of the guide wire begins to return to its unconstrained shape, i.e., the distal end portion of the guide wire begins to curve or wind into its coil shape. Referring to FIG. 15a, the guide wire 170 is advanced and deployed into cancellous bone of the vertebral body 164 until the coil shape has the desired number of loops or windings.

Referring to FIG. 15b, after the guide wire 170 has achieved a desired deployed configuration, the introducer sheath 160 and cannula 168 can be retracted and removed from the system. At this stage, the coiled distal end portion 174 of the guide wire 170 is deployed within the vertebral body 164, and the proximal end portion 176 of the guide wire is extending out of the passageway 166 of the vertebral body. The proximal end portion 176 of the guide wire defines an insertion path or track for the implant 178, which can function as the access device in this application for intraosseous venous access and will be referred to as the bone access device. Alternatively, when desired, the introducer sheath and/or cannula can be left in place, and the access device 178 can be deployed into the vertebral body through the introducer sheath, the cannula or both.

One of the advantages of removing the introducer sheath and the cannula from the system is that such removal allows for a larger passageway into the vertebral body. The larger passageway makes it possible to employ access devices or implants having larger dimensions. Thus, when the introducer sheath and cannula are removed, the dimensions of the access device can be larger because the size of the access device is not constrained or controlled by the size of the introducer sheath or cannula.

As illustrated in FIG. 15b, the access device 178 is inserted over the proximal end portion (not shown) of the guide wire 170, and a pusher member 180 is placed over the guide wire behind or proximal the access device. As the pusher member 180 is advanced, it contacts the access device 178 and advances it forward or distally over the guide wire 170.

Referring to FIG. 15c, as the access device 178 is advanced forward (distally) over the guide wire 170, the guide wire guides the access device through the passageway 166 and into vertebral body 164. The distal end 172 of the access device can be tapered, ramped or otherwise shaped to aid in passing through tissue.

In the vertebral body, the access device 178 follows along the coil shaped portion of the guide wire 170. The side slots in the access device allow it to bend more easily and follow the contour of the guide wire. One advantage of this embodiment of the access device, as noted above, is that it can be inserted through a small access hole and a much larger three dimensional support structure, such as a multi-tiered arrangement or scaffolding, can be built within a limited or confined space between or within the tissue layers. For instance the access device 178 can be inserted through a small access hole and the access device formed one loop at the time by adding one thickness of the access device over another one.

After the access device 178 has been deployed, the guide wire 170 can be retracted from the access device and removed from the system. This can be accomplished by holding the pusher member 180 in place while retracting the guide wire 170 in a proximal direction. As noted earlier, although illustrated in the context of a vertebral body, the same or similar procedure may be employed to access other bones for insertion of a bone access device in accordance with the present subject matter to provide intraosseous access to the systemic venous system.

Although the present subject matter has been described with reference to the illustrated examples, this is solely for purposes of explanation and not limitation. It is understood that the present subject matter may have application in other circumstances or may be varied in detail without departing from the disclosure herein.

1. A method of providing intraosseous access to the systemic venous system of a living subject comprising (1) implanting a venous access system within the subject, the system comprising a bone access device including a drug discharge aperture, a port and a fluid flow path fluidly extending between the port and the access device, the implanting including: (a) inserting the access device into bone marrow space of a bone in the subject and (b) positioning the port at a subcutaneous location accessible by transcutaneous needle insertion.

2. The method of claim 1 including delivering drug from the port through the access device and into the bone.

3. The method of claim 1 in which the port includes a reservoir for holding a quantity of drug.

4. The method of claim 1 including aspirating fluid from the port.

5. The method of claim 1 including anchoring the device within the bone.

6. The method of claim 1 wherein the bone is a vertebral body and the drug is for treating metastases of the spine.

7. The method of claim 1 including injecting a drug transcutaneously into the port.

8. The method of claim 1 in which the access device includes an internal lumen in flow communication with the fluid flow path and a plurality of discharge apertures in the device in flow communication with the lumen.

9. The method of claim 1 including inserting the access device into a bone marrow cavity of a bone.

10. The method of claim 1 in which the bone is one of the vertebral body, the iliac crest, the pelvis, the femur, the shoulder blade and a long bone.

11. The method of claim 1 in which the fluid flow path comprises fluid flow tubing and the method includes connecting the fluid flow tubing to the access device after it is inserted into the bone.

12. A system for providing intraosseous access to the systemic venous system of a living subject, the system comprising a bone access device including a drug discharge aperture for implantation into a bone, a port including a needle penetrable septum for subcutaneous location in the subject and fluid flow tubing extending between and fluidly connecting the port and the access device.

13. The system of claim 12 in which the bone access device includes an elongated member configured for anchoring within a bone.

14. The system of claim 13 in which the elongated member has a generally helical configuration in situ.

15. The system of claim 12 in which the bone access device includes retention surfaces to restrict withdrawal from bone.

16. The system of claim 12 in which the bone access device includes a fluid passageway and a plurality of fluid discharge openings communicating with the passageway.

17. The system of claim 17 in which the bone access device includes a helical thread on an external surface thereof.

18. The system of claim 12 in which the bone access device is configured to be pushed into the marrow space.

19. The system of claim 12 in which the bone access device is configured to be advanced into the marrow space by rotation.

20. The system of claim 13 in which the elongated member is configured to be advanced into the marrow space in a generally straight configuration and curved in situ.
